(19) United States
(12) Patent Application Publication
(10) Pub. No.: US 2012/0294936 A1
(43) Pub. Date: Nov. 22, 2012

(54) REDUCED MASS METFORMIN FORMULATIONS

(75) Inventors: Admassu Abebe, Princeton, NJ (US); Kyle Martin, Princeton, NJ (US); Jatin M. Patel, Princeton, NJ (US); Peter Timmins, Wirral (GB)

(73) Assignees: ASTRAZENECA UK LIMITED, London (GB); BRISTOL-MYERS SQUIBB COMPANY, Princeton, NJ (US)

(21) Appl. No.: 13/509,206

(22) PCT Filed: Nov. 12, 2010
(86) PCT No.: PCT/US10/56525

§ 371 Date: Jul. 25, 2012

Related U.S. Application Data
(60) Provisional application No. 61/261,049, filed on Nov. 13, 2009.

(51) Int. Cl. A61K 31/155; A61K 9/14; A61K 9/52; A61K 31/17; A61P 3/06; A61K 38/28; A61K 38/26; A61P 3/10; A61P 3/04; A61K 9/28; A61K 31/426

(52) U.S. Cl. 424/452; 514/635; 424/465; 424/490; 514/592; 514/369; 514/6.5; 514/7.2

(57) ABSTRACT

The present invention relates to metformin extended release (XR) formulations with improved compactability to provide reduced mass tablets, granulations, and capsules.

### FIELD OF THE INVENTION

The present invention relates to metformin extended release (XR) formulations with improved compactability to provide reduced mass tablets, granulations, and capsules.

### BACKGROUND OF THE INVENTION

Type II diabetes is the most common form of diabetes accounting for 90% of diabetes cases. Over 100 million people worldwide have type-2 diabetes (nearly 17 million in the U.S.) and the prevalence is increasing dramatically in both the developed and developing worlds. Type-II diabetes is a lifelong illness, which generally starts in middle age or later part of life, but can start at any age. Patients with type-2 diabetes do not respond properly to insulin, the hormone that normally allows the body to convert blood glucose into energy or store it in cells to be used later. The problem in type-2 diabetes is a condition called insulin resistance where the body produces insulin, in normal or even high amounts, but certain mechanisms prevent insulin from moving glucose into cells. Because the body does not use insulin properly, glucose rises to unsafe levels in the blood, the condition known as hyperglycemia.

Over time, sustained hyperglycemia leads to glucotoxicity, which worsens insulin resistance and contributes to dysfunction in the beta cells of the pancreas. The degree of sustained hyperglycemia is directly related to diabetic microvascular complications and may also contribute to macrovascular complications. In this way, hyperglycemia perpetuates a cycle of deleterious effects that exacerbate type 2 diabetes control and complications.

It is now widely accepted that glycemic control makes a difference in type II diabetes patients. The goal of diabetes therapy today is to achieve and maintain as near normal glycemia as possible to prevent the long-term microvascular and macrovascular complications associated with elevated glucose in the blood. Oral therapeutic options for the treatment of type II diabetes mellitus include compounds known as: sulfonylureas, biguanides (metformin), thiazolidinediones, and alpha-glucosidase inhibitors. The active agents from each class are generally administered to patients alone. However, once monotherapy becomes inadequate, combination therapy is an attactive and rational course of action for treating hyperglycemia despite the known side effect of weight gain associated with sulfonylurea and thiazolidinone therapies.

Metformin is disclosed in U.S. Pat. No. 3,174,901 and is currently marketed in the U.S. by Bristol-Myers Squibb Company in the form of its hydrochloride salt as GLUCOPHAGE® XR containing either 500 or 750 mgs of active ingredient. The Glucophage formulations contain sodium carboxymethyl cellulose, hydroxypropyl methylcellulose, and magnesium stearate as inactive ingredients.

Metformin XR formulations that improve compactability, without affecting the amount of active ingredient, are desirable because these formulations provide smaller tablets (granulations/capsules) that are more convenient for patients to use orally. Smaller tablets improve patient acceptability and compliance. Accordingly, the present invention provides extended release metformin formulations with improved compactability that results in smaller tablet size.

### SUMMARY OF THE INVENTION

The present invention provides extended release pharmaceutical formulations comprising metformin, one or more binders, one or more release modifiers, one or more glidants, one or more lubricants, and optionally a coating. These formulations have improved compactability that provide tablets, granulations, and capsules with reduced size and mass.

In another aspect, the present invention provides methods of treating diseases or disorders associated with SGLT2 activity comprising administering to a mammal in need of such treatment a therapeutically effective amount of a reduced mass metformin XR formulation, alone, or in combination with one or more anti-diabetics. The formulations of the present invention can be administered to mammals, preferably humans, for the treatment of a variety of conditions and disorders associated with SGLT2 activity including, but not limited to, treating or delaying the progression or onset of diabetes (including Type I and Type II diabetes), impaired glucose tolerance, insulin resistance, and diabetic complications, such as nephropathy, retinopathy, neuropathy and cataracts, hyperglycemia, hyperinsulinemia, hypercholesterolemia, dyslipidemia, elevated blood levels of free fatty acids or glycerol, hyperlipidemia, hypertriglyceridemia, obesity, wound healing, tissue ischemia, atherosclerosis and hypertension. The formulations of the present invention can also be utilized to increase the blood levels of high density lipoprotein (HDL). In addition, the conditions, diseases, and maladies collectively referenced to as “Syndrome X” or Metabolic Syndrome as detailed in Johannsson, J. Clin. Endocrinol. Metab., 82, 727-34 (1997), can be treated employing the formulations of the present invention.

In another aspect, the present invention provides methods for preparing the reduced mass metformin XR formulations.

### DETAILED DESCRIPTION OF THE INVENTION

The present invention provides reduced mass metformin XR formulations that comprise silicon dioxide or colloidal silicon dioxide with reduced amounts of hydroxypropyl methylcellulose. Hydroxypropyl methylcellulose is reduced from about 27% to about 18% while maintaining similar release rates. Further, the compactability of the reduced mass metformin XR granulation is improved significantly by adding silicon dioxide (e.g., Syloid®) or colloidal silicon dioxide (e.g., Aerosil 200®). Accordingly, the formulations of the present invention provide reduced mass tablets, granulations, and capsules that improve patient acceptability and compliance and can be used in diabetic fixed dose combination therapies.

In another aspect, the present invention provides pharmaceutical formulations comprising metformin hydrochloride, sodium carboxymethyl cellulose, hydroxypropyl methylcellulose, silicon dioxide or colloidal silicon dioxide, and magnesium stearate. The formulation is optionally coated wherein Opadry® II is the preferred coating.

In another aspect, the present invention provides pharmaceutical formulations comprising about 72-82% metformin hydrochloride, about 3-5% sodium carboxymethyl cellulose, about 15-22% hydroxypropyl methylcellulose 2208, about 0.75-1.25% silicon dioxide or about 0.25-0.75% colloidal silicon dioxide, and about 0.1-0.5% magnesium stearate. The formulation is optionally coated wherein Opadry® II is the preferred coating.

In another aspect, the present invention provides pharmaceutical formulations comprising about 76.6% metformin hydrochloride, about 3.84% sodium carboxymethyl cellulose, about 18% hydroxypropyl methylcellulose 2208, about 1% silicon dioxide, and about 0.53% magnesium stearate. The formulation is optionally coated wherein Opadry® II is the preferred coating.

In another aspect, the present invention provides metformin XR formulations in combination with one or more: anti-diabetics; anti-hyperglycemic agents; hypolipidemic/lipid lowering agents; anti-obesity agents; anti-hypertensive agents appetite suppressants; insulin secretagogues, insulin sensitizers, glucokinase activators, glucocorticoid antagonist, fructose 1,6-bis phosphatase inhibitors, AMP kinase activators, modulators of the incretin pathway such as incretin secretagogues such as GPR119 or GPR40 agonists, incretin mimics such as Byetta, and incretin potentiators, bile acid sequestrants or bile acid receptor agonists such as TGR5 agonists, dopamine receptor agonists such as Cycloset, aldose reductase inhibitors PPARγ agonists, PPARα agonists, PPARδ antagonists or agonists, PPARα/γ dual agonists, 11-β-HSD-1 inhibitors, dipeptidyl peptidase IV (DPP4) inhibitors other than saxagliptin, SGLT2 inhibitors other than dapagliflozin, glucagon-like peptide-1 (GLP-1), GLP-1 agonists, and PTP-1B inhibitors. Other substances that can be included in combination with metformin XR include weight loss agents acting to decreasing food intake such as sibutrimine, CB1 antagonists, 5HT2C agonists, MCHR1 antagonists, and agents which decrease nutrient absorption (such as lipase inhibitors (Orlistat)), and agents which increase energy expenditure such as thyromimetics, or slow GI motility such as amylin mimetics or ghrelin antagonists.

Examples of suitable anti-diabetic agents for use in combination with the formulations of the present invention include, but are not limited to, alpha glucosidase inhibitors (acarbose or miglitol), insulins (including insulin secretagogues or insulin sensitizers), meglitinides (repaglinide), sulfonylureas (glimepiride, glyburide, gliclazide, chlorpropamide and glipizide), biguanide/glyburide combinations (Glucovance®), thiazolidinediones (e.g., troglitazone, rosiglitazone and pioglitazone), PPAR-alpha agonists, PPAR-gamma agonists, PPAR alpha/gamma dual agonists, glycogen phosphorylase inhibitors, inhibitors of fatty acid binding protein (aP2), GPR-119 modulators, GPR 40 modulators, glucokinase inhibitors, glucagon-like peptide-1 (GLP-1) and other agonists of the GLP-1 receptor, SGLT2 inhibitors other than dapagliflozin, and dipeptidyl peptidase IV (DPP4) inhibitors other than saxagliptin.

Other suitable thiazolidinediones include, but are not limited to, MCC-555 (disclosed in U.S. Pat. No. 5,594,016, Mitsubishi), faraglitazar (GI-262570, Glaxo-Wellcome), englitazone (CP-68722, Pfizer) or darglitazone (CP-86325, Pfizer; isaglitazone, MIT/Johnson& Johnson), reglitazar (JTT-501, (JPNT/Pharmacia & Upjohn), rivoglitazone (R-119702, Sankyo/WL), liraglutide (NN-2344, Dr. Reddy/NN), and (Z)-1,4-bis-4-[(3,5-dioxo-1,2,4-oxadiazolidin-2-yl-methyl)]phenoxybut-2-ene (YM-440, Yamanouchi).

Examples of PPAR-alpha agonists, PPAR-gamma agonists and PPAR alpha/gamma dual agonists include, but are not limited to, muraglitazar, peliglitazar, tesaglitazar AR-HO39242 (Astra/Zeneca), GW-501516 (Glaxo-Wellcome), KRP297 (Kyorin Merck), as well as those disclosed by Murakami et al, “A Novel Insulin Sensitizer Acts As a Coligand for Peroxisome Proliferation-Activated Receptor Alpha (PPAR alpha) and PPAR gamma Effect on PPAR alpha Activation on Abnormal Lipid Metabolism in Liver of Zucker Fatty Rats”, Diabetes 47, 1841-1847 (1998); WO 01/21602 and in U.S. Pat. No. 6,414,002 and U.S. Pat. No. 6,653,314, the disclosures of which are incorporated herein by reference in their entireties, employing dosages as set out therein. In one embodiment, the compounds designated as preferred in the cited references are preferred for use herein.

Suitable aP2 inhibitors include, but are not limited to, those disclosed in U.S. application Ser. No. 09/391,053, filed Sep. 7, 1999, and in U.S. Pat. No. 6,548,529, the disclosures of which are incorporated herein by reference in their entireties, employing dosages as set out therein.

Suitable DPP4 inhibitors include, but are not limited to, sitagliptin and vildagliptin, as well as those disclosed in WO99/38501, WO99/46272, WO99/67279 (PROBIODRUG), WO99/67278 (PROBIODRUG), WO99/61431 (PROBIODRUG), NVP-DPP728A (1-[[[2-[(5-cyanopyridin-2-yl)amino]ethyl]amino]acetyl]-2-cyano-(S)-pyrrolidine) (Novartis) as disclosed by Hughes et al, Biochemistry, 38(36), 11597-11603, 1999, TSL-225 (tryptophyl-1,2,3,4-tetrahydroisoquinoline-3-carboxylic acid (disclosed by Yamada et al, Bioorg. & Med. Chem. Lett. 8 (1998) 1537-1540), 2-cyanopyrrolidides and 4-cyanopyrrolidides, as disclosed by Ashworth et al, Bioorg. & Med. Chem. Lett., Vol. 6, No. 22, pp 1163-1166 and 2745-2748 (1996), the compounds disclosed in U.S. application Ser. No. 10/899,641, all of which are incorporated herein by reference in their entireties, employing dosages as set out in the above references.

Suitable SGLT2 inhibitors contemplated by the present invention include sergliflozin, remogliflozin, remogliflozin etabonate, canagliflozin, BI-10773 and BI-44847, ASP-1941, R-7201, LX-4211, YM-543, AVE 2268, TS-033 or SGL-0100, and the compounds disclosed in U.S. Pat. No. 7,589,193, WO2007007628, EP2009010, WO200903596, US2009030198, U.S. Pat. No. 7,288,528 and US 2007/0197623, herein incorporated by reference in their entirety for any purpose. The following SGLT2 inhibitors, are preferred

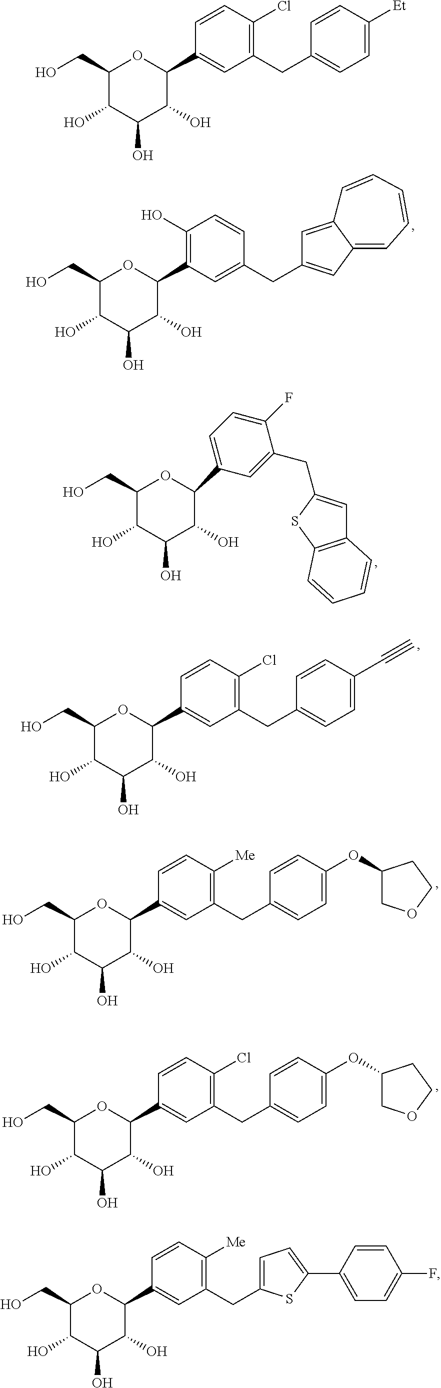

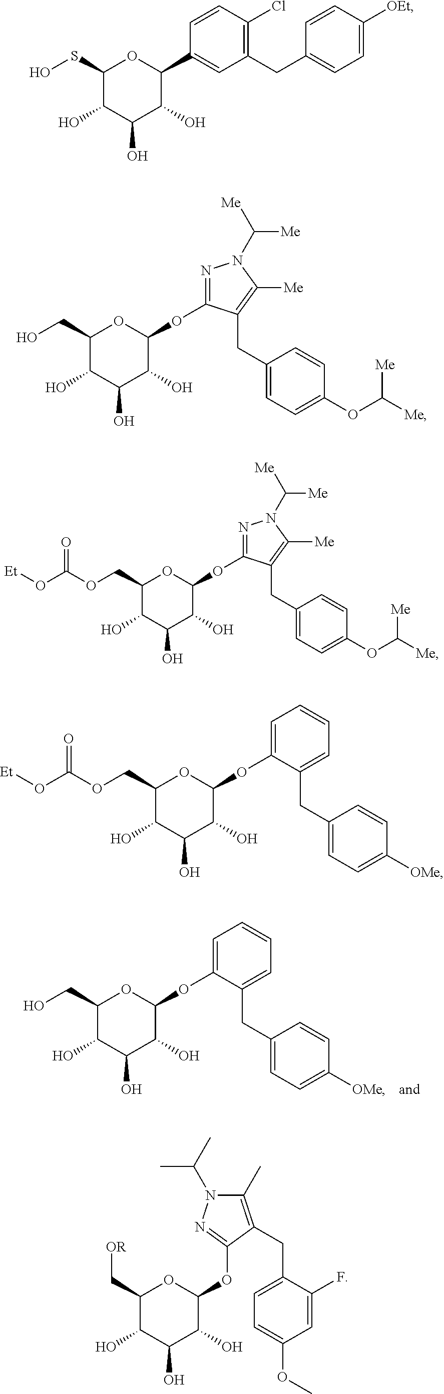

Suitable meglitinides include nateglinide (Novartis) or KAD1229 (PF/Kissei).

Examples of suitable anti-hyperglycemic agents for use in combination with the formulations of the present invention include, but are not limited to, glucagon-like peptide-1 (GLP-1) such as GLP-1(1-36) amide, GLP-1(7-36) amide, GLP-1(7-37) (as disclosed in U.S. Pat. No. 5,614,492, incorporated herein by reference in its entirety), as well as exenatide (Amylin/Lilly), LY-315902 (Lilly), MK-0431 (Merck), liraglutide (NovoNordisk), ZP-10 (Zealand Pharmaceuticals A/S), CJC-1131 (Conjuchem Inc), and the compounds disclosed in WO 03/033671, incorporated herein by reference in its entirety.

Examples of suitable hypolipidemic/lipid lowering agents for use in combination with the formulations of the present invention include one or more MTP inhibitors, HMG CoA reductase inhibitors, squalene synthetase inhibitors, fibric acid derivatives, ACAT inhibitors, lipoxygenase inhibitors, cholesterol absorption inhibitors, ileal Na⁺/bile acid co-transporter inhibitors, up-regulators of LDL receptor activity, bile acid sequestrants, cholesterol ester transfer protein (e.g., CETP inhibitors, such as torcetrapib (CP-529414, Pfizer) and JTT-705 (Akros Pharma)), PPAR agonists (as described above) and/or nicotinic acid and derivatives thereof. The hypolipidemic agent can be an up-regulator of LD2 receptor activity, such as 1(3H)-isobenzofuranone,3-(13-hydroxy-10-oxotetradecyl)-5,7-dimethoxy—(MD-700, Taisho Pharmaceutical Co. Ltd) and cholestan-3-ol,4-(2-propenyl)-(3a,4a,5a)—(LY295427, Eli Lilly). Preferred hypolipidemic agents include pravastatin, lovastatin, simvastatin, atorvastatin, fluvastatin, cerivastatin, atavastatin and rosuvastatin (ZD-4522), for example.

Examples of MTP inhibitors that can be employed as described above include, but are not limited to, those disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. Pat. No. 5,962,440, all of which are incorporated herein by reference in their entireties.

Examples of HMG CoA reductase inhibitors that can be employed in combination with the formulations of the invention include, but are not limited to, mevastatin and related compounds, as disclosed in U.S. Pat. No. 3,983,140, lovastatin (mevinolin) and related compounds, as disclosed in U.S. Pat. No. 4,231,938, pravastatin and related compounds, such as disclosed in U.S. Pat. No. 4,346,227, simvastatin and related compounds, as disclosed in U.S. Pat. Nos. 4,448,784 and 4,450,171. Other suitable HMG CoA reductase inhibitors that can be employed herein include, but are not limited to, fluvastatin, disclosed in U.S. Pat. No. 5,354,772, cerivastatin, as disclosed in U.S. Pat. Nos. 5,006,530 and 5,177,080, atorvastatin, as disclosed in U.S. Pat. Nos. 4,681,893, 5,273,995, 5,385,929 and 5,686,104, atavastatin (Nissan/Sankyo's nisvastatin (NK-104)), as disclosed in U.S. Pat. No. 5,011,930, rosuvastatin (Shionogi-Astra/Zeneca (ZD-4522)), as disclosed in U.S. Pat. No. 5,260,440, and related statin compounds disclosed in U.S. Pat. No. 5,753,675, pyrazole analogs of mevalonolactone derivatives, as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No. 0142146 A2, and quinoline and pyridine derivatives, as disclosed in U.S. Pat. Nos. 5,506,219 and 5,691,322. In addition, phosphinic acid compounds useful in inhibiting HMG CoA reductase, such as those disclosed in GB 2205837, are suitable for use in combination with the formulations of the present invention. All of the cited references are incorporated herein by reference in their entireties.

Examples of squalene synthetase inhibitors suitable for use herein include, but are not limited to, α-phosphono-sulfonates disclosed in U.S. Pat. No. 5,712,396, those disclosed by Biller et al., J. Med. Chem., 1988, Vol. 31, No. 10, pp. 1869-1871, including isoprenoid (phosphinyl-methyl)phosphonates, as well as other known squalene synthetase inhibitors, for example, as disclosed in U.S. Pat. Nos. 4,871,721 and 4,924,024 and in Biller, S. A., Neuenschwander, K., Ponpipom, M. M., and Poulter, C. D., Current Pharmaceutical Design, 2, 1-40 (1996). Other squalene synthetase inhibitors suitable for use herein include the terpenoid pyrophosphates disclosed by P. Ortiz de Montellano et al, J. Med. Chem., 1977, 20, 243-249; the farnesyl diphosphate analog A and presqualene pyrophosphate (PSQ-PP) analogs as disclosed by Corey and Volante, J. Am. Chem. Soc., 1976, 98, 1291-1293; phosphinylphosphonates reported by McClard, R. W. et al, J.A.C.S., 1987, 109, 5544; and cyclopropanes reported by Capson, T. L., PhD dissertation, June, 1987, Dept. Med. Chem. U of Utah, Abstract, Table of Contents, pp 16, 17, 40-43, 48-51, Summary. All of the cited references are incorporated herein by reference in their entireties.

Examples of fibric acid derivatives that can be employed in combination the formulations of the invention include, but are not limited to, fenofibrate, gemfibrozil, clofibrate, bezafibrate, ciprofibrate, clinofibrate and the like, probucol, and related compounds, as disclosed in U.S. Pat. No. 3,674,836, bile acid sequestrants, such as cholestyramine, colestipol and DEAE-Sephadex (Secholex®, Policexide®), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives, such as disclosed in U.S. Pat. No. 4,759,923, quaternary amine poly(diallyldimethylammonium chloride) and ionenes, such as disclosed in U.S. Pat. No. 4,027,009, and other known serum cholesterol lowering agents. In one embodiment, the fibric acid derivative is probucol or gemfibrozil. All of the cited references are incorporated herein by reference in their entireties.

Examples of ACAT inhibitors that can be employed in combination with the formulations of the invention include, but are not limited to, those disclosed in Drugs of the Future 24, 9-15 (1999), (Avasimibe); “The ACAT inhibitor, C1-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. The first water-soluble ACAT inhibitor with lipid-regulating activity Inhibitors of acyl-CoA:cholesterol acyltransferase (ACAT). Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62, or TS-962 (Taisho Pharmaceutical Co. Ltd). All of the cited references are incorporated herein by reference in their entireties.

Examples of suitable cholesterol absorption inhibitors for use in combination with the formulations of the invention include, but are not limited to, SCH48461 (Schering-Plough), as well as those disclosed in Atherosclerosis 115, 45-63 (1995) and J. Med. Chem. 41, 973 (1998), incorporated herein by reference in its entirety.

Examples of suitable ileal Na⁺/bile acid co-transporter inhibitors for use in combination with the formulations of the invention include, but are not limited to, compounds as disclosed in Drugs of the Future, 24, 425-430 (1999), incorporated herein by reference in its entirety.

Examples of lipoxygenase inhibitors that can be employed in combination with the formulations of the invention include, but are not limited to, 15-lipoxygenase (15-LO) inhibitors, such as benzimidazole derivatives, as disclosed in WO 97/12615, 15-LO inhibitors, as disclosed in WO 97/12613, isothiazolones, as disclosed in WO 96/38144, and 15-LO inhibitors, as disclosed by Sendobry et al “Attenuation of diet-induced atherosclerosis in rabbits with a highly selective 15-lipoxygenase inhibitor lacking significant antioxidant properties”, Brit. J. Pharmacology (1997) 120, 1199-1206, and Cornicelli et al., “15-Lipoxygenase and its Inhibition: A Novel Therapeutic Target for Vascular Disease”, Current Pharmaceutical Design, 1999, 5, 11-20. All of the cited references are incorporated herein by reference in their entireties.

Examples of suitable anti-hypertensive agents for use in combination with the formulations of the present invention include, but are not limited to, beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates. All of the cited references are incorporated herein by reference in their entireties.

Examples of suitable anti-obesity agents for use in combination with the formulations of the present invention include, but are not limited to, beta 3 adrenergic agonists, lipase inhibitors, serotonin (and dopamine) reuptake inhibitors, thyroid receptor beta drugs, 5HT2C agonists, (such as Arena APD-356); MCHR1 antagonists, such as Synaptic SNAP-7941 and Takeda T-226926, melanocortin receptor (MC4R) agonists, melanin-concentrating hormone receptor (MCHR) antagonists (such as Synaptic SNAP-7941 and Takeda T-226926), galanin receptor modulators, orexin antagonists, CCK agonists, NPY1 or NPY5 antagonist, NPY2 and NPY4 modulators, corticotropin releasing factor agonists, histamine receptor-3 (H3) modulators, 11-beta-HSD-1 inhibitors, adinopectin receptor modulators, monoamine reuptake inhibitors or releasing agents, ciliary neurotrophic factors (CNTF, such as AXOKINE® by Regeneron), BDNF (brain-derived neurotrophic factor), leptin and leptin receptor modulators, cannabinoid-1 receptor antagonists (such as SR-141716 (Sanofi) or SLV-319 (Solvay)), and anorectic agents.

Beta 3 adrenergic agonists that can be optionally employed in combination with formulations of the present invention include, but are not limited to, AJ9677 (Takeda/Dainippon), L750355 (Merck), CP331648 (Pfizer,) or other known beta 3 agonists, as disclosed in U.S. Pat. Nos. 5,541,204, 5,770,615, 5,491,134, 5,776,983 and 5,488,064, all of which are incorporated herein by reference in their entireties.

Examples of lipase inhibitors that can be employed in combination with formulations of the present invention include, but are not limited to, orlistat and ATL-962 (Alizyme).

Serotonin (and dopamine) reuptake inhibitors (or serotonin receptor agonists) that can be employed in combination with the formulations of the present invention include, but are not limited to, BVT-933 (Biovitrum), sibutramine, topiramate (Johnson & Johnson) and axokine (Regeneron).

Examples of thyroid receptor beta compounds that can be employed in combination with formulations of the present invention include, but are not limited to, thyroid receptor ligands, such as those disclosed in WO 97/21993 (U. Cal SF), WO 99/00353 (KaroBio) and WO 00/039077 (KaroBio), incorporated herein by reference it their entireties.

Examples of monoamine reuptake inhibitors that can be employed in combination with the formulations of the present invention include, but are not limited to, fenfluramine, dexfenfluramine, fluvoxamine, fluoxetine, paroxetine, sertraline, chlorphentermine, cloforex, clortermine, picilorex, sibutramine, dexamphetamine, phentermine, phenylpropanolamine and mazindol.

Anorectic agents that can be employed in combination with the formulations of the present invention include, but are not limited to, topiramate (Johnson & Johnson), dexamphetamine, phentermine, phenylpropanolamine and mazindol.

The aforementioned patents and patent applications are incorporated herein by reference.

Where any of the formulations of the invention are used in combination with other therapeutic agent(s), the other therapeutic agent(s) can be used, for example, in the amounts indicated in the Physician's Desk Reference, as in the cited patents and patent applications set out above, or as otherwise known and used by one of ordinary skill in the art.

### Example 1

Commercially available extended release formulations containing metformin (1000 mgs) were prepared as described below.

| Ingredient | % w/w | amount (mg) |
|---|---|---|
| Metformin HCl | 68.97 | 1000 |
| Sodium Carboxymethyl Cellulose | 3.45 | 50.01 |
| Purified water or water for injection | — | q.s.^{(a)} |
| Hydroxypropyl Methylcellulose 2208 | 27.10 | 393 |
| Magnesium Stearate | 0.48 | 7.00 |
| Total Metformin XR | 100 | 1450 |

Metformin HCl, 0.5% magnesium stearate, and sodium carboxymethyl cellulose were combined and mixed into a high shear granulator for one minute. Purified water, using a nozzle, was added with stirring for one minute. The wet granulated material was passed through a mill and then dried until the moisture content was 1.0% or less. The dried material containing metformin HCl, 0.5% magnesium stearate, and sodium carboxymethyl cellulose was passed through a mill and discharge into polyethylene-lined drums to provide milled metformin 1 g bulk granulation.

Hydroxypropyl methylcellulose 2208 USP (100,000 centipoise) (methocel K100M Premium) was added to a bin blender and mixed for 60 revolutions. The material was passed through a mill and discharge to provide milled hydroxypropyl methylcellulose 2208 USP.

Metformin (milled 1 g bulk granulation), hydroxypropyl methylcellulose 2208 USP (milled), hydroxypropyl methylcellulose 2208 USP (unmilled), and magnesium stearate were added to a bin blender and mixed for 60 revolutions. The mixed material was discharge into polyethylene-lined drums to provide metformin extended release 1 g bulk granulation.

### Example 2

Extended release formulations containing reduced mass metformin (1000 mgs) were prepared as described below.

| Ingredient | % w/w | amount (mg) |
|---|---|---|
| Metformin HCl | 76.62 | 1000 |
| Sodium Carboxymethyl Cellulose | 3.84 | 50.01 |
| Purified water or water for injection | — | q.s.^{(a)} |
| Hydroxypropyl Methylcellulose 2208 | 18.01^{(b)} | 235 |
| Silicon Dioxide | 1.00^{(c)} | 13 |
| Magnesium Stearate | 0.53 | 7 |
| Total Metformin XR | 100 | 1305 |

^{(a)}refers to the quantity sufficient to make the granulation composition 100% w/w
^{(b)}The range is 15%-27%
^{(c)}The range is 0.75%-1.25%

Metformin HCl, 0.5% magnesium stearate, and sodium carboxymethyl cellulose were combined and mixed into a high shear granulator for one minute. Purified water, using a nozzle, was added with stirring for one minute. The wet granulated material was passed through a mill and then dried until the moisture content was 1.0% or less. The dried material containing metformin HCl, 0.5% magnesium stearate, and sodium carboxymethyl cellulose was passed through a mill and discharge into polyethylene-lined drums to provide milled metformin 1 g bulk granulation.

Metformin (milled 1 g bulk granulation), hydroxypropyl methylcellulose 2208 USP (100,000 centipoise) (methocel K100M Premium), and silicon dioxide were added to a bin blender and mixed for 120 revolutions. Magnesium stearate was added, and after 60 revolutions, the material was discharge into polyethylene-lined drums to provide reduced mass metformin extended release 1 g bulk granulation.

The granulation process used to prepare commercially available metformin hydrochloride extended release (XR) tablets (750 mg), described in Example 1, is a wet granulation process. The commercial formulation contains about 27% hydroxypropyl methyl cellulose (HPMC), a slow release polymer, and about 69% active ingredient. The commercially prepared granulation is compressed to a tablet that weighs 1088 mgs to provide 750 mgs of active ingredient. This commercial process, therefore, requires compression of a tablet weighing 1450 mgs to deliver 1000 mgs of metformin. Tablets of this size may be difficult for certain patients to swallow.

Formulations of the present invention have been developed to reduce the size of the metformin hydrochloride XR tablet weight by reducing the amount of HPMC in the formulation while maintaining comparable release rates. Formulations comprising about 18% HPMC have similar release rates to the commercial formulations containing 27% HPMC. The 9% decrease in polymer level provides a lower size/weight tablet but also reduces the compactability of the granulation. The resultant lower compactability was overcome by the addition of silicon dioxide or colloidal silicon dioxide. Accordingly, metformin XR formulations of the present invention, containing silicon dioxide and reduced levels of HPMC, provide tablets with reduced mass (10%) and size while maintaining the appropriate metformin release rates.

We claim:

1. A metformin pharmaceutical formulation comprising (1) metformin; (2) one or more binders; (3) one or more release modifiers; (4), one or more glidants; (5) one or more lubricants; and (6) optionally a coating; wherein the pharmaceutical formulation is an extended release formulation in the form of a reduced mass tablet, stock granulation, or capsule.

2. The pharmaceutical formulation according to claim 1 comprising (1) metformin hydrochloride; (2) sodium carboxymethyl cellulose; (3) hydroxypropyl methylcellulose; (4) silicon dioxide or colloidal silicon dioxide; (5) magnesium stearate; and (6) optionally Opadry® II.

3. The pharmaceutical formulation according to claim 2 comprising (1) about 72-82% metformin hydrochloride; (2) about 3-5% sodium carboxymethyl cellulose; (3) about 15-22% hydroxypropyl methylcellulose 2208; (4) about 0.75-1.25% silicon dioxide or about 0.25-0.75% colloidal silicon dioxide; and (5) about 0.1-0.5% magnesium stearate.

4. The pharmaceutical formulation according to claim 2 comprising (1) about 76.6% metformin hydrochloride; (2) about 3.84% sodium carboxymethyl cellulose; (3) about 18% hydroxypropyl methylcellulose 2208; (4) about 1% silicon dioxide; and (5) about 0.53% magnesium stearate.

5. The pharmaceutical formulation according to claim 4 wherein there is a coating and the coating is Opadry® II.

6. The pharmaceutical formulation according to claim 2 comprising (1) about 1000 mgs of metformin hydrochloride; (2) about 50 mgs of sodium carboxymethyl cellulose; (3) about 235 mgs of hydroxypropyl methylcellulose 2208; (4) about 13 mgs of silicon dioxide; and (5) about 7 mgs of magnesium stearate.

7. The pharmaceutical formulation according to claim 6 wherein there is a coating and the coating is Opadry® II.

8. A pharmaceutical formulation comprising (1) about 76.6% metformin hydrochloride; (2) about 3.84% sodium carboxymethyl cellulose; (3) about 18% hydroxypropyl methylcellulose 2208; (4) about 1% silicon dioxide; (5) about 0.53% magnesium stearate; (6) an antidiabetic other than metformin; and (7) optionally a coating.

9. The pharmaceutical formulation according to claim 8 wherein the anti-diabetic is a sulfonylurea, thiazolidinedione, alpha glucosidase inhibitor, meglitinide, glucagon-like peptide (GLP) agonist, insulin, amylin agonist, fructose 1,6-bis phosphatase inhibitor, insulin secretagogue, insulin sensitizer, glucokinase activator, glucocorticoid antagonist, AMP kinase activator, modulators of the incretin pathway such as incretin secretagogue, incretin mimic, incretin potentiator, bile acid sequestrant or bile acid receptor agonist such as TGR5 agonist, dopamine receptor agonist, aldose reductase inhibitor, PPARγ agonist, PPARα agonist, PPARδ antagonist or agonist, PPARα/γ dual agonist, 11-β-HSD-1 inhibitor, dipeptidyl peptidase IV (DPP4) inhibitor other than saxagliptin, SGLT2 inhibitor other than dapagliflozin, glucagon-like peptide-1 (GLP-1), GLP-1 agonist, or PTP-1B inhibitor.

10. A pharmaceutical formulation comprising (1) about 76.6% metformin hydrochloride; (2) about 3.84% sodium carboxymethyl cellulose; (3) about 18% hydroxypropyl methylcellulose 2208; (4) about 1% silicon dioxide; (5) about 0.53% magnesium stearate; (6) a weight loss agent; and (7) optionally a coating.

11. The pharmaceutical formulation according to claim 10 wherein the weight loss agent is sibutrimine, a CB1 antagonist, a 5HT2C agonist, a MCHR1 antagonist, Orlistat, a thyromimetic, an amylin mimetic, or a ghrelin antagonist.

12. A pharmaceutical combination comprising the pharmaceutical formulation according to claim 3 and at least one additional therapeutic agent selected from the group consisting of anti-obesity agents; anti-diabetic agents, appetite suppressants; cholesterol/lipid-lowering agents, and HDL-raising agents.

13. The pharmaceutical combination according to claim 12, wherein the antidiabetic agent is selected from the group consisting of SGLT2 inhibitors other than dapagliflozin, DPPIV inhibitors other than saxagliptin, a thiazolidinedione, metformin in an immediate release form, a sulfonylurea, alpha glucosidase inhibitor, meglitinide, glucagon-like peptide (GLP) agonist, insulin, amylin agonist, fructose 1,6-bis phosphatase inhibitor, insulin secretagogue, insulin sensitizer, glucokinase activator, glucocorticoid antagonist, AMP kinase activator, modulators of the incretin pathway such as incretin secretagogue, incretin mimic, incretin potentiator, bile acid sequestrant or bile acid receptor agonist such as TGR5 agonist, dopamine receptor agonist, aldose reductase inhibitor, PPARγ agonist, PPARα agonist, PPARδ antagonist or agonist, PPARα/γ dual agonist, 11-β-HSD-1 inhibitor, glucagon-like peptide-1 (GLP-1), GLP-1 agonist, PTP-1B inhibitor, sibutrimine, CB1 antagonist, 5HT2C agonist, MCHR1 antagonist, Orlistat, thyromimetic, amylin mimetic, or ghrelin antagonist.
